(19) United States
(12) Patent Application Publication
(10) Pub. No.: US 2009/0030341 A1
(43) Pub. Date: Jan. 29, 2009

(54) SAMPLE RELEASE SYSTEM

(75) Inventors: Tushar A. Kshirsagar, Woodbury, MN (US); Jeffrey D. Smith, Croix, MN (US); Paul J. Cobian, Woodbury, MN (US); Tera M. Nordby, Woodbury, MN (US); Manjiri T. Kshirsagar, Woodbury, MN (US); Patrick A. Mach, Shorewood, MN (US); Bryan S. Behun, White Bear Lake, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/829,493

(22) Filed: Jul. 27, 2007

(51) Int. Cl. A61B 10/00

(52) U.S. Cl. 600/572; 436/174

(57) ABSTRACT

Devices to process a sample are configured to releasably attach a sample acquisition device to a drive mechanism that provides rotational and/or vibrational motion to the sample acquisition device. The devices can be used in sample processing methods to dislodge sample materials from the sample acquisition device. Sample processing methods using rotational or vibrational motion may further include the use of a liquid medium into which the sample is released. Sample processing methods may further include the detection of an analyte in the sample.

### BACKGROUND

Sample acquisition devices, such as swabs, are generally used in many industries for collecting a sample of material from a sample source. The sample acquisition device can include a hollow shaft including a distal end and a proximal end, and a sample-collecting region. The sample-collecting region is typically a structured surface, such as a porous medium, attached to the distal end of the hollow shaft. The distal end and proximal end may be open or include an opening. In the medical industry, the sample acquisition device may be used to gather a sample of biological material from a nose, ear, throat, or other sample source (e.g., a wound). Specifically, the shaft may be handled to position the porous medium in contact with the nose, ear, throat, or other sample source. In the food service industry, the shaft of the sample acquisition device may be handled to position the porous medium in contact with a food preparation surface, a food container, or environmental surface, and the like. The samples collected by the sample acquisition device may then be analyzed for the presence of, for example, an organism. The analysis may incorporate an assay.

Prior to the analysis of the sample, the sample is typically transferred from the sample acquisition device in order to place the sample in condition for analysis. In some methods, the sample acquisition device may be placed in contact with a slide or other laboratory apparatus in order to transfer at least some of the sample to the slide or other laboratory apparatus. In other methods, a fluid, such as a buffer solution, may be introduced into the proximal end of the hollow shaft of the sample acquisition device. The fluid then flows through the hollow shaft and exits through an opening at the distal end, contacting the sample as the liquid exits the hollow shaft and passes through the porous medium.

The efficiency of release of the sample from the porous medium can affect the overall sensitivity of the analyses. Thus, some methods use a mechanical vortex to wash the sample off the sample acquisition device. Although mechanical vortexing facilitates the release of analytes from a sample acquisition device, the method may be subject to operator variability. Additionally, the need for a mechanical vortex instrument may preclude certain point-of-care applications.

For these reasons, there is a need for a method that can be used to release a sample from a sample acquisition device consistently, efficiently, and without the need for highly-skilled technicians.

### SUMMARY

In one aspect, the present invention includes a method of releasing a sample from a sample acquisition device. In this aspect, the method comprises providing a sample acquisition device having a sample-collecting region with a sample disposed thereon, a liquid medium, and a device comprising a rotary drive. The method further comprises attaching the sample acquisition device to the rotary drive, contacting the sample-collecting region with the liquid medium, and activating the rotary drive.

In another aspect, the present invention includes a method of releasing a sample from a sample acquisition device. In this aspect, the method comprises providing a sample acquisition device having a sample-collecting region with a sample disposed thereon, a liquid medium, and a device comprising a vibrational drive. The method further comprises attaching the sample acquisition device to the vibrational drive, contacting the sample-collecting region with the liquid medium, and activating the vibrational drive.

In another aspect, the present invention includes a device for the detection or identification of analytes in a sample. In this aspect, the device comprises a sample preparation module to dislodge sample from sample acquisition devices. The sample preparation module comprises a rotary drive configured for the releasable attachment of a sample acquisition device and a detection system to detect the presence of an analyte or a component thereof

In another aspect, the present invention includes a device for the detection or identification of analytes in a sample. In this aspect, the device comprises a sample preparation module to dislodge sample from sample acquisition devices. The sample preparation module comprises a vibrational drive configured for the releasable attachment of a sample acquisition device and a detection system to detect the presence of an analyte or a component thereof.

In another aspect, the present invention includes a device for processing a sample. The device comprises a vibrational drive configured for the releasable attachment of a sample acquisition device and a sample acquisition device attached thereto.

### Definitions

As used herein, the term “rotary drive” refers to an apparatus that can be used to turn an object in a rotational motion around an axis. The apparatus may be powered by a variety of mechanisms, such as manually, electrically, or hydraulically, for example. The object rotated by the rotational drive may be linked directly or indirectly to the drive. Alternatively, the drive may provide a force, such as, for example, air or water pressure or a magnetic field, which causes and/or controls the rotational motion of the object. The elements of the linkage through which the rotational force is transferred to the object are considered to be a part of the rotary drive.

As used herein, the term “vibrational drive” refers to a powered apparatus that can be used to cause an object to vibrate, oscillate, undulate, or pulsate. The apparatus may be powered by a variety of mechanisms, such as manually, electrically, or hydraulically, for example. The object vibrated by the vibrational drive may be linked directly or indirectly to the drive. Alternatively, the drive may provide a force, such as, for example, air or water pressure or a magnetic field, which causes and/or controls the rotational motion of the object. The elements of the linkage through which the vibrational force is transferred to the object are considered to be a part of the vibrational drive.

As used herein, the term “sample acquisition device” refers to a device that is used to obtain a sample of material. The sample may comprise solid materials, liquid materials, or combinations thereof.

As used herein, the term “analyte” refers to any material to be detected and/or quantified in a sample. Analytes may be simple or complex chemical materials, such as atoms or molecules and may either be organic or inorganic. Analytes also include various molecules (e.g., Protein A) or epitopes of molecules (e.g., different binding sites of Protein A), or whole cells, such as eukaryotic cells, tissue, or microorganisms. These include components of eukaryotic cells, tissue, cell walls (e.g., cell-wall proteins such as protein A), external cell components (e.g., capsular polysaccharides and cell-wall carbohydrates), internal cell components (e.g., nucleic acids, cytoplasmic membrane proteins), etc.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “comprises” and variations thereof do not have a limiting meaning where those terms appear in the description and claims.

As used herein, “a”, “an”, “the”, “at least one”, and “one or more” are used interchangeably. Thus, for example, a sample acquisition device that comprises “a” sample-collecting region can be interpreted to mean that the sample acquisition device can include “one or more” sample-collecting regions.

The term “and/or” means one or all of the listed elements or a combination of any two or more of the listed elements.

Also herein, the recitations of numerical ranges by endpoints include all numbers subsumed within the range (e.g., 1 to 5 includes 1, 1.5, 2, 2.75, 3, 3.80, 4, 5, etc.).

The above summary of the present invention is not intended to describe each disclosed embodiment or implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout several views.

FIG. 1 shows a frontal perspective view of a device, with sample acquisition device attached thereto, according to one embodiment of the present invention;

FIG. 2a shows a perspective view of a sample acquisition device with rotational motion according to one embodiment of the present invention;

FIG. 2b shows a perspective view of a sample acquisition device with rotational and orbital motion according to one embodiment of the present invention;

FIG. 3 shows a perspective view of a sample acquisition device with vibrational motion according to one embodiment of the present invention; and

FIG. 4 shows a perspective view of a sample acquisition device with vibrational motion according to an alternative embodiment of the present invention.

### DETAILED DESCRIPTION

The present disclosure concerns devices and methods for preparing a sample, such as a biological sample, for detecting an analyte, such as a bacterium. The disclosure further concerns the release of sample materials from a sample acquisition device, such as a swab, so that the sample materials can be available for analysis by a number of methods which are discussed below.

Sample acquisition devices, such as a swab, are routinely used to collect samples for chemical, biochemical, biological, or microbiological analyses of various materials or surfaces. The sample acquisition devices comprise a sample-collecting region, which is contacted with the material or surface to be analyzed. The sample-collecting region can comprise a molded material, such as plastic; a nonwoven fibrous material, such as rayon; nylon, cotton or polyester, or a foam material, such as polyurethane foam or a cellulose sponge. The most commonly used sample acquisition devices comprise a sample-collecting region, comprised of nonwoven fibrous materials, located at the tip of the device. The sample-collecting region accumulates sample material by, for example, adsorption, absorption, physical entrapment, or combinations thereof.

At least one drawback to the use of sample acquisition devices comprising nonwoven fibers or foams is the inherent variability of such materials. For example, the fibers can exhibit various sizes, shapes, density, and spatial arrangement. Foams typically comprise hollow cells formed in a variety of shapes, sizes and spatial arrangement. This inherent variability can affect the ease from which a sample is dislodged from the material. Furthermore, the release of the sample from the sample acquisition device can be affected by the skill and experience of the lab technician performing the procedure. This may introduce an additional element of variability to a test procedure. The present invention provides a number of devices and methods by which these elements of variability may be minimized, and thereby provide more consistent and/or efficient release of a sample from a sample acquisition device.

One aspect of the invention includes a device to improve the efficiency and/or consistency of the release of sample material from a sample acquisition device, without the requirement for the use of an electrically-powered machine, such as a vortex mixer. Another aspect of the invention includes a device to improve the homogeneity of a sample of material for analysis, without requiring a separate, unattached, electrically-powered instrument, such as a vortex mixer. Another aspect of the invention includes methods and devices to reduce the variability of the release of analytes, such as microorganisms, from individual sample acquisition devices of similar constructions. Accordingly, such devices and/or methods can be used to prepare a sample for analysis by a number of techniques, which are discussed in further detail below.

The inventive devices are relatively simple and allow a sample of material to be collected, prepared, and, optionally, tested for an analyte at or near the sample source. Rather than transferring the sample of material to an off-site laboratory for preparation and analysis, the present invention allows an operator to obtain a sample of material from a sample source, prepare the sample for analysis, and then test for the presence of an analyte at or near the sample source. This helps to decrease the waiting time necessary for a test result. Of course, the inventive device can also be used in a laboratory or other off-site setting.

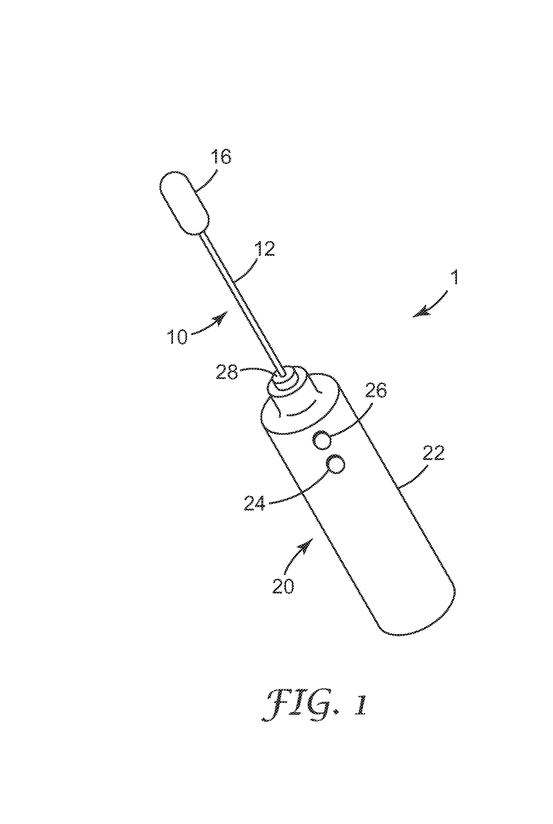

An exemplary device in accordance with the present invention is shown in FIG. 1. This perspective view shows the device 1 is comprised of a drive 20 and a sample acquisition device 10. The drive 20 comprises a housing 22, with either a rotary drive or vibrational drive motor (not shown) contained therein, and a connector 28 which is configured for the releasable attachment of the sample acquisition device 10 to the drive 20. The drive 20 may optionally comprise an actuator switch 24 to activate the motor and/or a regulator switch 26 making the speed of the rotary or vibrational drive 20 adjustable. Other aspects of the drive 20 are discussed below.

The housing 22 affords protection for the drive mechanism and motors from contamination with materials that may interfere with their functioning. The housing 22 may be constructed from various materials, such as metal or plastics. In certain embodiments, the housing 22, with the drive motor contained therein, is relatively small and, thus, portable. Optionally, the housing 22 may comprise a handle (not shown), so that the device may be hand-held during transport or use.

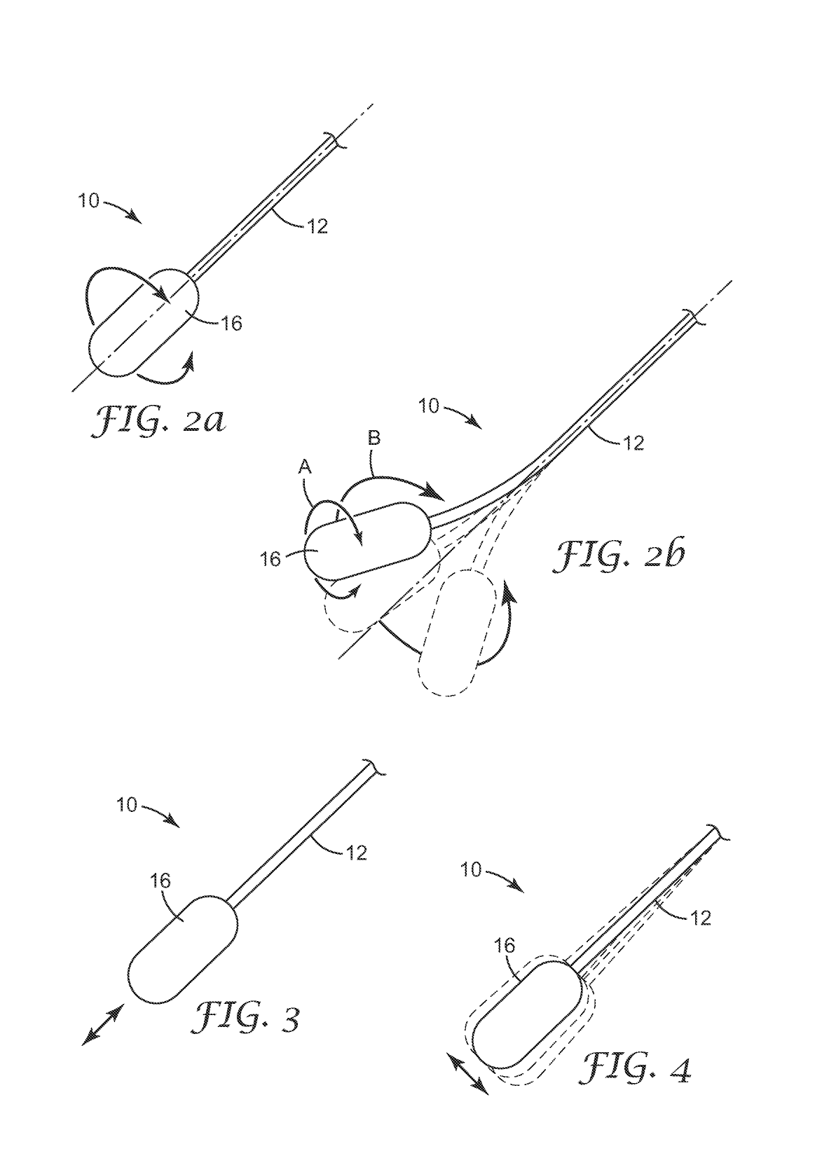

FIG. 2a shows a perspective view of the sample acquisition device 10 subjected to rotational motion according to the present invention. The sample acquisition device 10 is comprised of a shank 12 and a sample-collecting region 16. The dashed line indicates the apparent axis of rotation, which runs essentially parallel to the shank 12 of the sample acquisition device 10. The arrow adjacent to the sample-collecting region 16 indicates that the rotational motion may be provided in either a clockwise or counterclockwise motion. In some embodiments, the rotational motion can be alternated between a clockwise and a counterclockwise motion.

It should be noted that, depending upon the length of the shank 12, the flexibility of the material from which the shank 12 is constructed, the mass of the sample-collecting region 16, and the rotational speed, the rotation of the sample acquisition device 10 around the axis of rotation may also impart a secondary “orbiting” motion of the sample-collecting region 16. That is, in addition to rotating around the imaginary axis, (the dashed line in FIG. 2b) of the shank 12, the sample-collecting region 16 may also orbit around the axis of rotation, creating a motion similar to stirring. These rotational and orbital motions are shown in FIG. 2b by the arrows labeled “A” and “B”, respectively. The orbital motion of the sample-collecting region 16 may enhance sample release in a liquid medium, if present, by creating additional movement of the liquid medium.

FIG. 3 shows a perspective view of the sample acquisition device 10 subjected to vibrational motion according to the present invention. The sample acquisition device 10 is comprised of a shank 12 and a sample-collecting region 16. The arrow adjacent to the sample-collecting region 16 indicates the vibrational motion, which is substantially parallel to the shank 12 of the sample acquisition device 10, imparted by the drive (not shown).

FIG. 4 shows a perspective of a sample acquisition device 10 subjected to a vibrational motion according to the present invention. The sample acquisition device 10 is comprised of a shank 12 and a sample-collecting region 16. The arrow adjacent to the sample-collecting region 16 indicates the vibrational motion, which is substantially perpendicular to the shank 12 of the sample acquisition device 10, imparted by the drive (not shown).

### Rotational and Vibrational Drive Motors

Motors providing rotational motion of a relatively small object, such as a sample acquisition device, are well known. A manually operable device for holding and rotating candy is described in U.S. Pat. No. 5,957,746 and is incorporated in its entirety herein by reference. FIGS. 6-8 of that patent illustrate how a pivotable trigger can be used to drive a set of gears to cause the rotation of a shaft to which a candy sucker may be attached. U.S. Pat. Nos. 5,209,692 and 6,183,336 disclose battery-powered devices to rotate candy and both patents are incorporated in their entirety herein by reference. Both patents describe how a small electric motor, coupled to a set of gears, can be used to rotate a shaft to which a candy sucker may be attached. In certain embodiments, the electric motor can be controlled by a switch. The switch can be a type which can be fixed in the “on” or “off position or one that makes electrical contact only when held in the “on” position. That is, the switch may be biased or spring-loaded toward the “off” position. In some embodiments, the rotational motion may be coupled to a vibrational motion, such as the coupled rotational and vibrational motions exemplified in a hammer drill, for example, which is used for drilling concrete or other hard materials. With no changes or relatively minor changes, it would be possible to adapt such devices or to construct similar devices to attach and to rotate sample acquisition devices. The devices may be designed to operate at a constant rotational speed. Alternatively, a rheostat may be added to such devices in order to obtain variable speed control for the rotational drive motor. Additionally, the gearing may be adjusted to provide for higher rotational speeds and/or a wide range of variable rotational speeds, motions and/or directions.

Motors for providing vibrational motion of a relatively small object are known. U.S. Pat. No. 6,085,850 and U.S. Patent Publication No. 2003/0047039 A1, which are incorporated in their entirety herein by reference, describe methods and devices to convert rotary motion into reciprocating motion using cam action drives. U.S. Pat. No. 5,515,930, which is incorporated in its entirety herein by reference, describes methods and devices to convert pressurized air into bidirectional oscillating movement. With no changes or relatively minor changes, it would be possible to adapt such devices or to construct similar devices to attach and vibrate sample acquisition devices, such as swabs. The device may be designed to operate at a constant vibrational speed. Alternatively, a controller may be added to such devices in order to obtain variable speed control for the vibrational drive motor. Additionally, the gearing may be adjusted to provide for higher vibrational speeds and/or a wide range of variable vibrational speeds, motions and/or directions.

### Microorganisms and Analytes

Microorganisms of particular interest for analytical purposes include prokaryotic and eukaryotic organisms, particularly Gram positive bacteria, Gram negative bacteria, fungi, bacterial or fungal spores, protozoa, mycoplasma, yeast, viruses, and even lipid-enveloped viruses. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, Escherichia coli O157 and multiple drug resistant Gram negative rods (MDR).

Gram positive and Gram negative bacteria are of particular interest for analytical purposes because there are a number of organisms within those groups that are known to be pathogenic to humans. Of even more interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, nucleic acid, etc., responsible for antibiotic resistance.

Analytes for detecting the organisms of interest include, for example, cell-wall proteins such as protein A and microbial surface components recognizing adhesive matrix molecules (MSCRAMMs) such as fibrinogen-binding proteins (e.g., Clumping Factor), fibronectin-binding proteins, collagen-binding proteins, heparin/heparin-related polysaccharides binding proteins, and the like. Protein A and Clumping Factor, such as fibrinogen-binding proteins and clumping factors A, B, and Efb, are also particularly useful in methods of detecting the presence of Staphylococcus aureus. Other external cell components of interest include capsular polysaccharides and cell-wall carbohydrates (e.g., teichoic acid and lipoteichoic acid).

### Samples and Sampling Techniques

Species of interest can be analyzed in a test sample that may be derived from any source, such as a physiological fluid, e.g., blood, saliva, ocular lens fluid, tears, vitreous humor, synovial fluid, cerebral spinal fluid, pus, sweat, exudate, urine, feces, mucus, lactation milk, or the like. Further, the test sample may be derived from a body site, e.g., wound, skin, nares, scalp, nails, etc. The samples may consist substantially of solid, semi-solid, gelatinous, or liquid material, alone or in various combinations.

Clinical samples of particular interest include mucus-containing samples, such as nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Other samples of particular interest include, for example, food processing contact surfaces, non-food contact surfaces, drains, walls, door handles, processed food, and raw materials.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like. Samples can also include cultured cells.

The art describes various patient sampling techniques for the detection of microbes, such as S. aureus. Such sampling techniques are suitable for the methods of the present invention as well. For example, it is common to obtain a sample from wiping the nares of a patient. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

The art further describes various methods to prepare samples containing mucus or biofilms for microbiological analyses. For example, nasal samples may contain biological materials, such as mucus which may interfere with the detection of microbes in the samples. In certain instances, mucolytic agents, such as N-acetylcysteine, can be added to the sample to dissolve the mucus and/or disperse the microorganisms in the sample. Alternatively, certain enzymes, such as proteases, glycosidases, or nucleases can be used to partially or completely digest certain polymers, such as proteins or polysaccharides, which may cause undue adherence of a sample to a sample acquisition device. In some embodiments, enzymes and/or mucolytic agents may be used in conjunction with rotational or vibrational motion to facilitate the release of a sample from a sample acquisition device.

In dental specimens, biofilms such as dental plaque can interfere with the detection and/or quantitation of microbes associated therewith. In certain instances, the plaque has been dispersed using ultrasonic vibration. However, excessive ultrasound can result in killing of subpopulations in plaque, which can affect the estimates of microbes present in the sample (W. H. Bowen, 1987, Advances in Dental Research, vol. 1, pages 88-91).

A wide variety of swabs or other sample acquisition devices are commercially available, for example, from Puritan Medical Products Co. LLC, Guilford, Me., under the trade designation PURE-WRAPS or from Copan Diagnostics, Inc. Corona, Calif., under the trade designation MICRORHEOLOGICS nylon flocked swab. A sample acquisition device such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

Sample acquisition devices of various lengths are available and the length may generally be selected based upon the intended use. For example, surface sampling methods may allow the use of a sample acquisition device with a relatively short shank, such as about 2 to about 7 centimeters in length. Alternatively, deep sampling methods may require the use of a sample acquisition device with a relatively long shank, such as about 15 to about 23 centimeters or longer. The present invention includes methods employing sample acquisition devices with relatively long or relatively short shanks.

### Sample-Processing Methods

After a sample has been acquired using a sample acquisition device, the sample acquisition device (e.g., swab) can then be cultured directly, analyzed directly, or extracted (e.g., by rotation or vibration) in an appropriate solution. Such extraction (i.e., elution) solutions typically include water and can optionally include a buffer and at least one surfactant. An example of an elution buffer includes, for example, phosphate buffered saline (PBS), which can be used in combination, for example, with TWEEN 20 or PLURONIC L64. The test sample (e.g., liquid) may be subjected to treatment prior to further analysis. Treatment options include concentration, precipitation, filtration, centrifugation, distillation, dialysis, dilution, inactivation of natural components, addition of reagents, chemical treatment, etc.

In certain embodiments, the extraction may be performed by attaching the swab to a device with a rotary drive or a vibrational drive, contacting the swab with a liquid medium, such as an elution buffer, and activating the rotary drive. In some embodiments, the sample-collecting region of the swab is completely immersed in the liquid medium. The drive may be activated to dislodge the sample from the sample acquisition device. The efficiency of sample release will be related to several factors, such as rotational speed, vibrational frequency and/or amplitude, and the amount of time that the rotational or vibrational force is applied to the sample acquisition device.

The drive may be activated for at least about 5 seconds, at least 15 seconds, at least 30 seconds, or at least 60 seconds. When a rotary drive is present, the speed of the rotary drive may be preselected. In certain embodiments, the rotational speed is preselected to at least about 57 rpm, at least about 297 rpm or at least about 1080 rpm. The angular velocity of the surface of the sample-collecting region is proportional to the rotational speed and, in general, a higher rotational speed will result in faster release of the sample material, as demonstrated in the examples described below. When a vibrational drive is present, the frequency of the vibrational drive may be preselected. In certain embodiments, the vibrational frequency is preselected to at least about 7200 strokes per minute (spm).

In certain embodiments, the liquid medium comprises a reagent. Exemplary reagents may be added to the liquid medium to adjust and/or maintain the pH of the sample, disrupt cells, to facilitate the release sample material from the sample acquisition device, to facilitate detection of a target analyte in the sample or any combination of two or more reagents thereof. Reagents to adjust the pH of the sample may include buffering agents, such as sodium phosphate, potassium phosphate, TRIZMA, HEPES, sodium bicarbonate, buffered saline and the like. The liquid medium may comprise reagents for specimen preservation or transport, such as Amies or Stuart's transport media. The liquid medium may comprise reagents to disrupt cells, such as an enzyme, an alkali, a surfactant, or a chaotroph and such reagents may release target analytes such as a protein or nucleic acid from a cell to facilitate the detection of the target analyte. Enzymes for cell disruption include, for example, lysozyme, lysostaphin, trypsin, or protease K. In addition to facilitating the disruption of cell wall or cell membranes to release a target analyte, surfactants additionally may facilitate the release of sample material from the sample acquisition device. Nonlimiting examples of said surfactants include ionic surfactants, such as sodium dodecylsulfate or one or more of the following nonionic agents commonly available in surfactant tool kits: NINATE 411, Zonyl FSN 100, Aerosol OT 100%, GEROPON T-77, BIO-TERGE AS-40, STANDAPOL ES-1, Tetronic 1307, Surfynol 465, Surfynol 485, Surfynol 104PG-50, IGEPAL CA210, TRITON X-45, TRITON X-100, TRITON X305, SILWET L7600, RHODASURF ON-870, Cremophor EL, TWEEN 20, TWEEN 80, BRIJ 35, CHEMAL LA-9, PLURONIC L64, SURFACTANT 10G, SPAN 60, CREL.

In certain embodiments, the liquid medium may comprise functionalized particles, such as polymeric or magnetic beads to capture an analyte for detection. The particles may contain antibodies, polynucleotides, or other binding agents disposed thereon to facilitate the capture of analytes. In these embodiments, the vibrational or rotational motion can facilitate the mixing and capture of the analytes.

After dispersing the sample from the sample acquisition device into the liquid medium, the sample may be analyzed for an analyte, such as a microorganism. The samples may be analyzed by chemical methods, such as chromatography or mass spectrometry; immunological methods, such as ELISA, immunochromatography, immunofluorescent microscopy, immunoprecipitation, latex agglutination or hemagglutination; flow cytometry; growth-based detection; microscopy; fluorescent immunochromatography; acoustic wave sensors; or colorimetric detection using a polydiacetylene material. Alternatively, the samples may be analyzed by genetic methods, such as nucleic acid amplification (for example, PCR, real-time PCR, LCR, and NASBA), hybridization or sequencing.

In another embodiment, the sample is released from the sample acquisition device using rotational or vibrational motion according to the present disclosure in a sample processing device comprising an abrasion element. Sample processing devices comprising abrasion elements are disclosed in U.S. patent application Ser. No. ______ (Attorney Docket No. 62950US002), filed on even date herewith, and entitled “APPARATUS AND METHOD FOR RELEASING A SAMPLE OF MATERIAL.”

### Integrated Sample Preparation and Detection Systems

In the field of diagnostic microbiology, there are a number of instruments that are used to detect or identify target microorganisms. The instruments use a variety of technologies to detect the presence of whole organisms or subcellular component “analytes”, such as soluble proteins, lipoproteins, membrane-associated proteins, polypeptides oligopeptides, enzymes, cell wall-associated proteins, DNA, rRNA, mRNA, tRNA, oligonucleotides, adenosine triphosphate, polysaccharides, peptidoglycans, teichoic acids, and lipotechoic acids.

The target analytes may be detected by immunological methods, through a binding reaction with specific antibodies, or genetic material specifying the target analyte may be detected by any known means for detecting DNA or RNA, such as genetic amplification (e.g., PCR, RT-PCR, LCR, and NASBA) or hybridization techniques. Non-limiting examples of such instruments used for target analyte detection include those described in the following patent publications: U.S. Pat. Nos. 6,889,468 and 7,056,473, and U.S. Patent Publication Numbers 2004/0137634A1 and 2005/0130177A1.

Devices of the present invention may be incorporated as a sample preparation module into a device used to detect analytes or components thereof. The sample preparation module comprise a rotational or a vibrational drive, as described above, attached or, optionally, removably attached to the instrument. The rotational or vibrational drive is configured to releasably attach a sample acquisition device. The sample acquisition device can be inserted into a tube or chamber and the rotational or vibrational drive can be activated to dislodge samples from the sample acquisition device.

The sample preparation module may be incorporated in an instrument with a detection system to detect the presence of an analyte or a component of an analyte in a sample. Suitable analytes for detection and exemplary detection instruments are described above.

### EXAMPLES

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

Sample-acquisition devices (swabs, part number SWB0405) were obtained from Medical Packaging Corporation (Camarillo, Calif.). The shanks of the swabs were cut to a length of approximately 6-7 cm (including the sample-collecting region) before use. The variable speed/reversible cordless electric drill (Craftsman model number 315.115400) used in the following examples was obtained from Sears (Sears Holding Corporation, Hoffman Estates, Ill.). The drill speed was controlled by adjusting the setting on the drill. Actual revolutions per minute (rpm) were measured with a digital tachometer. The swabs were attached to the drill by inserting the swab shank approximately 1 cm into the drill chuck and tightening until the swab was held firmly by the chuck. All experiments with the drill were performed using clockwise rotation of the swab.

A Model 290 electric engraver sold by the Robert Bosch Tool Corporation (Mount Prospect, Ill.) under the trade name DREMEL was used for the vibrational motion experiments described below. A piece of plastic tubing approximately 7.5 cm long was attached to the bit of the engraver and the swabs were attached to the tubing as described below.

Staphylococcus aureus ATCC 6538 was obtained from the American Type Culture Collection (Manassas, Va.).

### Example 1

### Preparation of Bacterial Cultures

An isolated colony of S. aureus ATCC was aseptically transferred to 10 mL of Tryptic Soy Broth (Hardy Diagnostics, Santa Maria, Calif.). The broth was incubated at 37° C. without shaking for 18-24 hours. The cells were washed twice using the following procedure: a) cells were pelleted by centrifugation at 10,000 rpm for 10 minutes (4° C.), b) the supernatant was discarded, and c) the cells were resuspended in an equal volume of phosphate buffered saline (PBS, 137 mM NaCl and 2.7 mM KCl in 10 mM phosphate buffer, pH 7.50). After the second wash, the cells were resuspended in an equal volume PBS. The final cell suspensions were diluted to working suspensions (approximately 10⁵ colony-forming units (cfu)) in PBS as described below.

### Example 2

### Release of Bacteria from a Swab by Rotational Motion

A suspension of washed S. aureus cells was prepared as described above. The suspension was diluted 1:7000 in PBS to obtain the working bacterial suspension. All experiments were performed using sterile disposable 12×75 mm polypropylene culture tubes containing 650 microliters of PBS. Bacteria were enumerated by using sterile spreaders to distribute 0.1 milliliter of the respective bacterial suspensions onto 0.5% sheep blood agar plates, and incubating the plates for 18 to 24 hours at 37° C. Duplicate plates were made for each bacterial suspension tested.

Two types of controls were performed: a) no swab controls and b) swab solution controls. The “no swab” controls were prepared by adding 10 microliters of the working bacterial suspension to tubes containing 650 microliters of PBS. The tubes were vortexed for about 5 seconds and the resulting suspensions were spread onto plates as described above. Three replicates of this control were performed. The “swab solution” control was prepared by placing a swab into a tube containing 650 microliters of PBS and vortexing the tube for approximately 5 seconds. The swab was removed and 10 microliters of the working bacterial suspension was added to the tube. The tubes were vortexed and the bacterial suspensions were spread onto plates as described above. Two replicates of this control were performed.

Two types of experimental tests were performed in this example to evaluate the release of bacteria from a swab: a) vortexing and b) rotational motion applied to the swab shank. In the latter tests, the rotational motion was applied to the swabs by the use of an electric drill. In the “vortex” experiments, 10 microliters of the working bacterial suspension were injected into the tip of the swab. The spiked swab was placed in a 12×75 mm culture tube containing 650 microliters of diluent. The swab was vortexed at the maximum setting (10) in the diluent for either 15 or 30 seconds. The swab was removed and the bacteria in the suspension remaining in the tube were enumerated as described above. In the “rotational motion” experiments, 10 microliters of the working bacterial suspension were injected into the tip of the swab. The shank of the spiked swab was attached to the drill chuck and the swab was placed into a 12×75 mm culture tube containing 650 microliters of diluent. The swab was rotated in the diluent under the conditions shown in Table 1. The swab was removed and the bacteria in the suspension remaining in the tube were enumerated as described above. Three replicates were performed for each “vortex” and “rotational motion” experimental condition.

TABLE 1
Conditions for the release of bacteria using rotational motion. All
experiments used a bacterial inoculum of approximately 10³ cfu.
The drill setting was the setting on the instrument. The speed was
adjusted by the pressure applied to the trigger and the rpm
were independently measured with a tachometer.

| | Drill | | | |
|---|---|---|---|---|
| Expt No | Setting | Speed | RPM | Time (sec) |
| 1 | Low | Low | 57 | 15 and 30 |
| 1 | Low | Medium | 107 | 15 and 30 |
| 1 | Low | High | 297 | 15 and 30 |
| 2 | High | Medium | 180 | 5, 10, and 15 sec |
| 2 | High | High | 1080 | 5, 10, and 15 sec |

Table 2 shows the release of bacteria from the swab using rotational motion. Two time points and three drill speeds were evaluated. The relatively high (>100%) results for most of the tests may be due to the release of a surfactant from the swabs into the PBS suspension. The surfactant, combined with the agitation, may have disaggregated clumps of S. aureus cells, leading to higher counts than the control with no swab present.

TABLE 2
Recovery of S. aureus from swabs subjected to rotational motion. The
recovery is listed as a percentage of the bacteria in the “no swab” control.
All data points represent the average of the replicate tests.

| Treatment | 15 seconds | 30 seconds |
|---|---|---|
| Vortex | 204 | 183 |
| Drill (slow) | 19 | 187 |
| Drill (medium) | 71 | 158 |
| Drill (fast) | 123 | 186 |
| Swab Solution Control | 298 | 331 |

### Example 3

### Release of Bacteria from a Swab by Rotational Motion

These experiments were conducted as described in Example 2 with the exceptions that a) treatment times as short as 5 seconds were examined and b) the drill speed was increased to 1,080 rpm in some experiments. The low drill speed (180 rpm) was achieved by depressing the trigger to one-half of the fully-depressed setting. The high drill speed (1080 rpm) was achieved by depressing the trigger fully. In this example, the suspension of washed S. aureus cells was diluted 1:6000 to produce a working bacterial suspension of approximately 10⁵ cfu/milliliter.

Table 3 shows the release of bacteria from the swab using rotational motion using a high-speed setting. Three time points (5, 10, and 15 seconds) were evaluated using the drill. Two drill speeds (180 and 1080 rpm) were evaluated.

TABLE 3
Recovery of S. aureus from rayon swabs. Each swab was inoculated with
approximately 10³ colony-forming units (cfu). The data presented in this
table is the average of replicate experiments and is expressed as cfu.

| Treatment | 5 seconds | 10 seconds | 15 seconds | 30 seconds |
|---|---|---|---|---|
| Drill (medium) | 2.34 × 10³ | 2.69 × 10³ | 3.12 × 10³ | NA |
| Drill (fast) | 3.36 × 10³ | 3.33 × 10³ | 3.20 × 10³ | NA |
| Vortex | 3.00 × 10³ | 2.84 × 10³ | 2.76 × 10³ | 2.81 × 10³ |
| Swab Solution | 3.50 × 10³ | 3.60 × 10³ | 3.86 × 10³ | 3.89 × 10³ |
| Control | | | | |

### Example 4

### Release of Bacteria from a Swab by Vibrational Motion

The working bacterial suspension was prepared as described in Example 1. Bacterial plate counts were conducted as described in Example 2. A “no swab” control was prepared as described in Example 2. Experiments using the Vortex mixer were performed as described in Example 2. In order to attach the swabs to the engraving tool, the end of the swab shank opposite the sample-collecting region was wrapped with approximately 1 cm of double-sided adhesive tape (3M Company, St. Paul, Minn.). The adhesive taped end was inserted into the plastic tubing attached to the engraving tool and swab was thereby attached firmly to the engraving tool. The engraving tool was set to the maximum speed for all experiments. The manufacturer reported that the no load vibration speed of the engraving tool is 7,200 strokes per minute (spm).

For the vibrational release experiments, the swab was inoculated with 10 microliters of working bacterial suspension as described above and was attached to the engraving tool. The swab was inserted into a PBS buffer solution and vibrated at the maximum speed (setting 5) for 5, 10, 15, or 30 seconds. The swab was removed from the buffer solution and the bacteria in the buffer solution were enumerated as described above. Each experimental data point is the mean of three replicates at each condition.

TABLE 4
Recovery of S. aureus from Rayon Swabs. The recovery is listed as a
percentage of the bacteria in the “no swab” control.

| Vibration Time | S. aureus Recovered (%) |
|---|---|
| 5 sec | 95 |
| 10 sec | 162 |
| 15 sec | 163 |
| 30 sec | 177 |
| 30 sec | 166 |
| Control | 203 |

The complete disclosures of all patents, patent applications, and publications that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method of releasing a sample from a sample acquisition device, the method comprising:
providing a sample acquisition device having a sample-collecting region with a sample disposed thereon, a liquid medium, and a device comprising a rotary drive;
attaching the sample acquisition device to the rotary drive;
contacting the sample-collecting region with the liquid medium; and
activating the rotary drive.

2. The method of claim 1 wherein contacting the sample-collecting region with the liquid medium comprises completely immersing the sample-collecting region in the liquid medium.

3. The method of claim 1 wherein the rotary drive is activated for at least 5 seconds.

4. The method of claim 1 wherein the rotary drive is activated for at least 60 seconds.

5. The method of claim 1 further comprising detecting an analyte in the sample.

6. The method of claim 5 wherein detecting the analyte comprises detecting a microorganism or a component thereof.

7. The method of claim 5 wherein detecting the analyte comprises detecting a bacterium, a virus, a yeast, a fungus, a spore, a protozoan or a component of any one of the foregoing.

8. The method of claim 5 wherein detecting the analyte comprises detecting a eukaryotic cell, a tissue, or a component of any one of the foregoing.

9. The method of claim 1 further comprising adjusting the speed of the rotary drive.

10. The method of claim 9 wherein the speed is adjusted to a preselected speed.

11. The method of claim 10 wherein the preselected speed is between about 57 revolutions per minute and about 1080 revolutions per minute.

12. The method of claim 10 wherein the preselected speed is between about 297 revolutions per minute and about 1080 revolutions per minute.

13. A device for the detection or identification of an analyte in a sample, the device comprising:
a sample preparation module to dislodge samples from sample acquisition devices, the module comprising a rotary drive; and
a detection system to detect the presence of the analyte or a component thereof;
wherein the rotary drive is configured for the releasable attachment of a sample acquisition device.

14. A device for processing a sample, the device comprising a vibrational drive configured for the releasable attachment of a sample acquisition device and a sample acquisition device attached thereto.

15. The device of claim 14 wherein the vibrational drive is a hand-held vibrational drive.

16. The device of claim 14 wherein the hand-held vibrational drive is battery-powered.

17. A method of releasing a sample from a sample acquisition device, the method comprising:
providing a sample acquisition device having a sample-collecting region with a sample disposed thereon, a liquid medium, and a vibrational drive;
attaching the sample acquisition device to the vibrational drive;
contacting the sample-collecting region with the liquid medium; and
activating the vibrational drive.

18. The method of claim 17 wherein contacting the sample-collecting region with the liquid medium consists of immersing the sample-collecting region in the liquid medium.

19. The method of claim 17 wherein the vibrational drive is activated for at least 5 seconds.

20. The method of claim 17 wherein the vibrational drive is activated for at least 60 seconds.

21. The method of claim 17 further comprising detecting an analyte in the sample.

22. The method of claim 21 wherein detecting the analyte comprises detecting a microorganism or a component thereof.

23. The method of claim 22 wherein detecting the analyte comprises detecting a bacterium, a virus, a yeast, a fungus, a spore, a protozoan or a component of any one of the foregoing.

24. The method of claim 17 wherein detecting the analyte comprises detecting a eukaryotic cell, a tissue, or a component of any one of the foregoing.

25. The method of claim 17 further comprising adjusting the speed of the vibrational drive.

26. The method of claim 25 wherein the speed is adjusted to a preselected speed.

27. A device for the detection of an analyte in a sample, the device comprising:
a sample preparation module to dislodge samples from sample acquisition devices, the module comprising a vibrational drive; and
a detection system to detect the presence of the analyte or a component thereof;
wherein the vibrational drive is configured for the releasable attachment of a sample acquisition device.
